(19) United States
(12) Patent Application Publication
(10) Pub. No.: US 2026/0015383 A1
(43) Pub. Date: Jan. 15, 2026

(54) PLANT PROTEIN ISOLATE AND PROCESS FOR PRODUCTION THEREOF

(71) Applicant: Louis Dreyfus Company Plant Proteins LLC, Livermore, CA (US)

(72) Inventors: Manoj Kumar, Livermore, CA (US); Prasanth Kumar Sasidharan Pillai, Livermore, CA (US)

(21) Appl. No.: 18/771,902

(22) Filed: Jul. 12, 2024

(51) Int. Cl. C07K 1/36; C07K 14/415

(52) U.S. Cl. CPC C07K 1/36; A23J 1/006; A23J 3/14; C07K 14/415

(57) ABSTRACT

The present disclosure provides an albumin enriched PPI extraction process and albumin enriched PPI produced by the process and use of albumin enriched PPI. The albumin enriched PPI extraction process comprises steps of a) protein extraction from aqueous solution comprising pea flour, b) separation of solids from the aqueous solution, producing a protein-rich liquid fraction, and c) precipitation of protein by use of Na, Ca, or Mg salt or combination thereof, thereby producing an albumin enriched PPI.

### BACKGROUND OF THE INVENTION

Plant protein research and development has grown recently due to the increasing demand for plant based proteins. Sources of non-animal protein are being pursued for various reasons, not the least of which is the inherent inefficiency of converting feed proteins to animal proteins. With the world population continuing to grow, along with increasing sentiment against the use of animals as primary food sources, continued increasing demand for plant sources of protein promise is expected. Efficient production of high quality plant protein that is both economical and results in the production of proteins with desirable functionality will benefit the world's increasing population and its evolving preferences.

### SUMMARY OF THE INVENTION

The present disclosure provides a plant protein isolate (PPI) extraction process. In some embodiments the plant source is faba bean (fava bean), from which faba bean protein isolate (FPI) is produced. In other embodiments, the plant source is mung bean, chickpea, lentil, black gram or pea. Generally, the present process may be applied to any pulse. As described throughout the present disclosure, the term plant protein isolate (PPI) may be substituted with the term fava bean protein isolate (FPI), chickpea protein isolate (CPI), mung bean protein isolate (MPI), lentil protein isolate (LPI), urad bean protein isolate (UPI) or pea protein isolate PPI) or, generally, pulse protein isolate (XPI), as each of the embodiments of the invention is described.

In certain embodiments the plant protein isolate (PPI) extraction process comprises a salt extraction step using sodium hexametaphosphate (SHMP) in a basic aqueous solution of plant product, providing a novel and economical process for extracting proteins from plant material. The plant protein isolate thus provided maintains native structure and has various improved functionalities over plant protein isolates produced by conventional methods. Generally, in this embodiment the plant protein isolate (PPI) extraction process comprises subsequent isoelectric precipitation (IEP) of the extracted plant protein.

In one aspect of the invention, the plant protein isolate (PPI) extraction process comprises extracting protein from a plant product in a neutral to basic aqueous solution comprising 0.2% to 1.5% SHMP to create a protein solution, separating the protein solution from the unsolublized plant solids and precipitating protein from the protein solution by lowering pH to produce a plant protein isolate.

In some aspects, the PPI extraction process comprises the steps of a) extracting protein from a plant product in a basic aqueous solution to create a protein solution, b) separating unsolublized plant solids from the aqueous solution, and c) precipitating protein from the protein solution by i. lowering the pH of the solution, and ii. Adding about 0.2% to about 5% SHMP to produce a plant protein isolate.

In certain aspects of the invention, the extracting is performed in the presence of about 0.2% to about 5% SHMP. In some aspects, the PPI extraction process comprises steps of a) extracting protein in an aqueous solution comprising plant product at a pH of 7.0 to 9.5, b) separating solids from the aqueous solution, producing a protein-rich liquid fraction, and c) precipitating protein at a pH about 3.0 to 7.0, at a temperature of about 15° C. to about 80° C. for about 5 to about 60 minutes using 0.2% to 1.5% SHMP.

In some embodiments, the a) extracting protein in a basic aqueous solution comprising plant product comprises extracting protein at a pH of about 7 to 10. In certain embodiments the extracting is performed at a temperature of about 15° C. to 60° C. In some embodiments the extracting is performed for about 30 to 60 minutes. In certain embodiments the extracting is performed in a solution with an initial concentration of about 2% to about 40% plant solids.

In some embodiments, the separating of step is performed by centrifugation. In certain embodiments, the centrifugation is performed at 5000 g-15000 g. In certain embodiments the separating step is by centrifugation for about 5 to about 30 minutes. In some embodiments, the centrifugation is performed at about 4° C. to 70° C. In other embodiments, the separating step is performed by decantation.

In some embodiments, the plant solids of step b) comprise starch and fiber and the liquid fraction of step b) comprises protein supernatant, vitamins, and minerals.

In some embodiments of the invention, the precipitating step is performed at about pH 3 to about pH 7. In certain embodiments the precipitating step is performed in the presence of about 0.2% to about 5% SHMP. In some embodiments the precipitating is performed at a temperature of about 30° C. to about 80° C. In certain embodiments the precipitating is formed for about 10 to about 60 minutes.

In some aspects, the PPI extraction process further comprises a step of d) washing the PPI from step c). In some embodiments, the washing of step d) is performed with water. In certain aspects the washing is performed at the precipitating pH. In some embodiments the washing is performed at about pH 3 to about pH 7. In certain embodiments the washing is performed 1-3 times.

In other aspects of the invention, the PPI extraction process further comprises diluting the PPI to create a slurry. In some embodiments the diluting is performed with water. In certain embodiments the diluting comprises adjusting the pH to about neutral.

In still other aspects, the PPI extraction process further comprises a step of homogenizing the PPI. In certain embodiments the PPI extraction process further comprises sterilizing the protein slurry. In some embodiments, the homogenizing step is performed using a high-pressure homogenizer. In some embodiments, the sterilizing step is performed using direct steam injection. In certain embodiments, the sterilizing is performed at a temperature of about 75° C. to about 140° C. for about 2 seconds to about 10 minutes.

In yet other aspects, the PPI extraction process further comprises drying the PPI. In some embodiments, the drying is performed by a freeze drying and/or spray drying.

The present disclosure provides PPI produced by the process of the present disclosure. In certain embodiments the disclosure the PPI is a faba bean PPI (FPI). In other embodiments the PPI is a chickpea protein isolate (CPI), mung bean protein isolate (MPI), lentil protein isolate (LPI), black gram protein isolate (UPI) or pea protein isolate PePI) or, generally, pulse protein isolate (XPI).

In some embodiments, the PPI has a protein content in the range of about 80 to about 90 wt % (N×6.25) on a dry basis.

In some embodiments, the PPI is substantially in its native form. In certain embodiments the PPI is FPI having thermal stability to about 97° C. In some embodiments the FPI has a thermal transition with a peak at about 96.6° C., as measured by differential scanning calorimetry (DSC). In some embodiments the FPI has a thermal transition with an onset of about 83.8° C., a peak at about 96.6° C. and an offset of about 107.3° C., as measured by DSC.

In some embodiments the PPI has an improved color profile as compared with PPI produced using conventional processes (conventional PPI).

In some embodiments, the PPI has protein solubility at room temperature and at pH 7 of about 73% to about 93%. In certain embodiments the PPI has protein solubility at room temperature and at pH 7 of about 80% to about 92%. In some embodiments, the PPI has protein solubility at room temperature and at pH 7 of about 80% to about 85%. In some embodiments, the PPI protein solubility at room temperature and at pH 7 is >80%. In certain embodiments the PPI protein solubility at room temperature and at pH 7 is >90%.

Certain aspects of the invention provides an emulsion comprising the PPI. Other aspects of the invention provides an foam comprising the PPI. Still other aspects of the invention provides an gel comprising the PPI.

In some aspects, the present disclosure further provides use of the PPI of the present disclosure in the production of a foam. In other aspects, the present invention provides use of the PPI in the production of a gel. In still other aspects, the present invention provides use of the PPI in the production of an emulsion. In some embodiments, the emulsion is made from a mixture of equal parts oil and water.

In some aspects, the present disclosure provides a foam comprising FPI of the present disclosure having about 400% foam expansion capacity (FE) at a concentration of 2-10% (w/w) in water when homogenized at 1000 rpm for 1 minute. In other aspects, the present disclosure provides a foam comprising FPI of the present disclosure having about 70% foam stability (FS) after 1 hour at a concentration of 2-10% (w/w) in water when homogenized at 1000 rpm for 1 minute. In certain embodiments of the invention, the FPI has 100% emulsion stability (ES) in cold storage after 5 days at a concentration of 2-10% (w/w) in a 50:50 oil in water emulsion.

In still other aspects, the present disclosure provides use of the PPI of the present disclosure in a food or beverage application. In some embodiments, the food or beverage application is selected from milk shake, protein bars, meat analogues, confectionary, condiments, mayonnaise, salad dressing, nutritional supplements and diary alternatives. In certain embodiments, the dairy alternative is selected from creamers, ice cream, yogurt, buttermilk and cheese.

In yet other aspects, the present disclosure provides a food or beverage comprising the PPI of the present disclosure.

### BRIEF DESCRIPTION OF THE DRAWINGS

FIG. 1 shows a diagram of the conventional PPI extraction process.

FIG. 2 shows a diagram of an embodiment of the PPI extraction process of the present disclosure.

FIG. 3 shows a diagram of a different embodiment of the PPI extraction process of the present disclosure.

FIG. 4 shows FPI produced using the PPI extraction process (A) and FPI produced using a conventional FPI extraction method.

FIGS. 5A and 5B show differential scanning calorimetry analysis of FPI produced using the process disclosed herein (5A) and of FPI obtained using a conventional FPI extraction method (5B).

FIG. 6 shows an emulsion prepared with FPI of the present invention (A) and FPI produced using a conventional FPI extraction method (B).

FIG. 7 shows a foam prepared with FPI of the present invention (A) and FPI produced using a conventional FPI extraction method (B).

### DETAILED DESCRIPTION OF THE INVENTION

The present disclosure provides a plant protein isolate (PPI) extraction process. More specifically, the plant protein isolate (PPI) extraction process comprises steps of a) extracting protein from a plant product in a basic aqueous solution comprising sodium hexametaphosphate, to create a protein solution b) separating the protein solution from unsolublized plant solids, and c) precipitating protein from the protein solution by lowering the pH, thereby producing PPI. The present disclosure also provides a plant protein isolate extraction process comprising the steps of a) extracting protein from a plant product in a basic aqueous solution, to create a protein solution b) separating the protein solution from unsolublized plant solids, and c) precipitating protein from the protein solution by adding sodium hexametaphosphate and lowering the pH, thereby producing PPI. The present disclosure also provides PPI produced by the disclosed process, including faba bean protein isolate (FPI) and other plant protein isolates from various plant sources.

Before the present processes, compositions and uses are described, it is to be understood that this invention is not limited to the particular methods or compositions described, which may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

The term “acidic” means having a pH lower than about 6.5.

The term “basic” means having a pH higher than about 7.5.

The term “neutral” means having a pH of about 6.5 to about 7.5.

“Extracting” or “extraction” means the removal or separation of one or more component(s) of a multicomponent composition. The concept of extracting a protein isolate from a plant product is well known in the present art.

“Precent foaming expansion (% FE)” describes the volume expansion of a protein solution produced by agitation, such as subjecting the solution to a homogenizer. Percent foaming expansion (% FE) is determined using the equation % FE=(Vfo/Vli)×100, where Vfo is the measured volume of agitated solution and Vli is the measured initial volume of liquid before agitation.

“Percent foaming stability (% FS)” is the measure of maintenance of foaming expansion over time. Percent foaming stability (% FS) is measured using the equation % FS=[(Vli−Vlt)/(Vli−Vlo)], where Vli is the measured initial volume of protein solution before agitation, Vlt is the volume of the protein solution one hour after agitation and Vlo is the volume of the protein solution immediately after agitation.

“Plant product” mean any plant or portion thereof containing protein. Typically the seed or bean of the plant. In certain embodiments, the plant product is the pulse of a legume, such as a pea, chickpea, lentil and beans, including soy beans, faba beans, mung beans and black grams. The plant product used in the processes described herein may be nominally processed, such as ground or flaked, or may have had oils extracted such as via pressing or by solvent. For legumes, the pulses are typically ground to a flour.

The term “plant protein isolate (PPI)” used herein refers to the final product of plant protein extraction from the plant product. It's a highly concentrated protein substance. Plant protein isolate is the extracted protein created through a process that separates protein from other elements of plant.

“Substantially in native form” in the context of proteins extracted from plants means that the proteins have not been denatured due to, for example, exposure to excessive heat, so that the 3-dimensional structure of the proteins are generally maintained in the form found in the pre-processed seed.

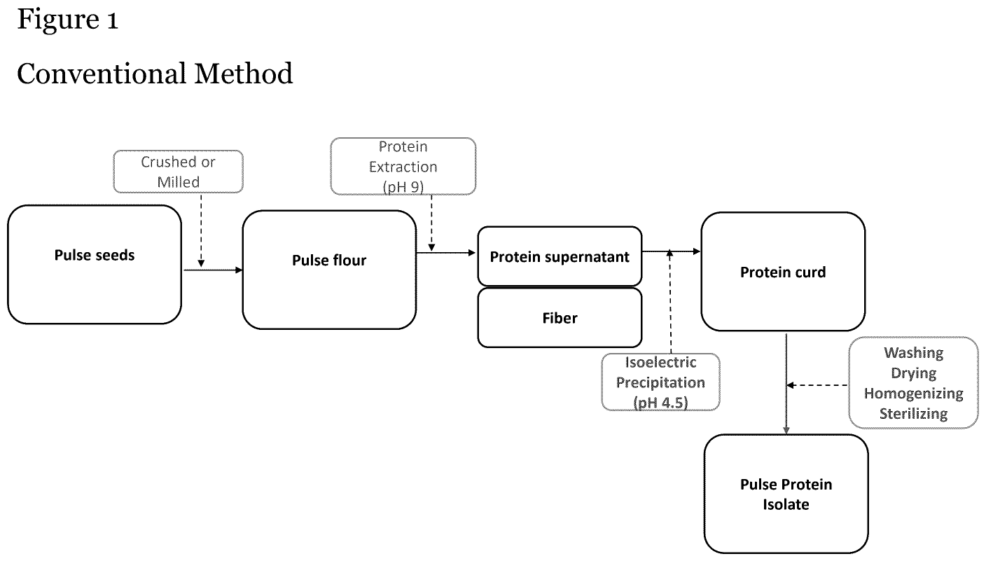

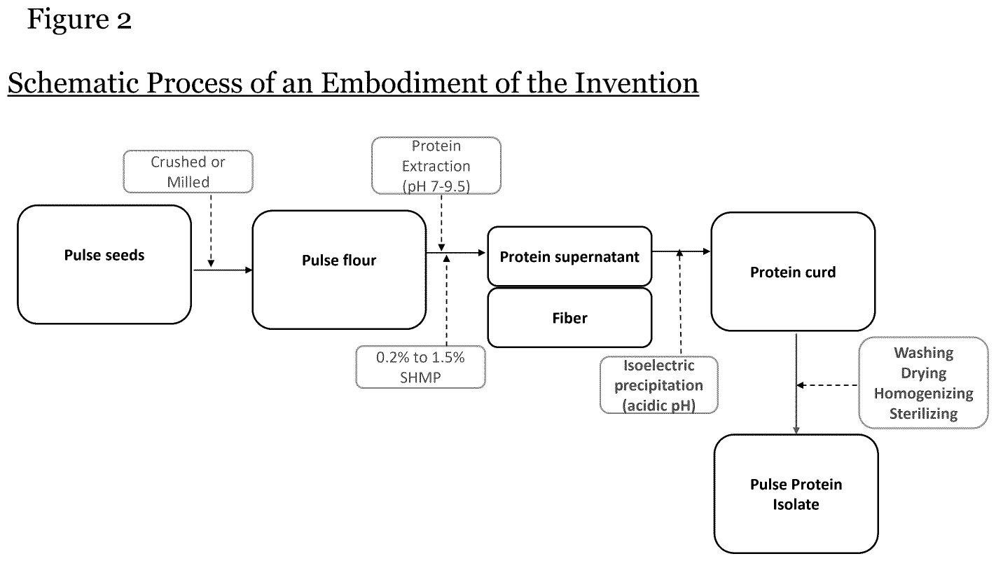

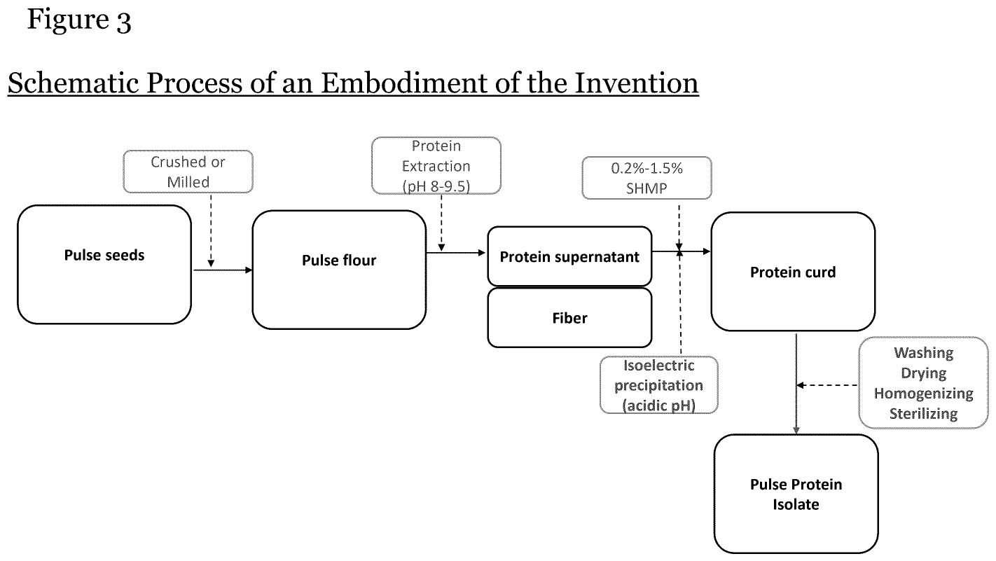

“Conventional PPI” used herein refers to PPI produced by the conventional PPI extraction process, which is well known in the art. The conventional PPI extraction process is depicted in FIG. 1.

### Plant Protein Isolate (PPI) Extraction Process

The present disclosure provides a plant protein isolate (PPI) extraction process. More specifically, the plant protein isolate (PPI) is prepared by the utilization of sodium hexametaphosphate (SMHP) in the protein isolate extraction process, either at the initial protein extraction step and/or at protein precipitation step. In some embodiments, the plant protein isolate (PPI) extraction process comprises a salt extraction step in combination with isoelectric precipitation (IEP) or alkaline extraction/isoelectric precipitation (AE/IEP).

In some aspects, the albumin enriched PPI extraction process comprises steps of a) extracting protein from a plant product in a basic aqueous solution comprising SHMP to produce a protein solution, b) separating the protein solution from unsolublized plant solids and c) precipitating protein from the protein solution by lowering the pH, thereby producing a plant protein isolate PPI. In certain aspects, the present disclosure provides a plant protein isolate extraction process comprising the steps of a) extracting protein from a plant product in a basic aqueous solution, to create a protein solution b) separating the protein solution from unsolublized plant solids, and c) precipitating protein from the protein solution by adding sodium hexametaphosphate and lowering the pH, thereby producing PPI.

### Starting Materials

Prior to preparing plant product for protein extraction, any kind of plant, including legume pulses, which contains protein as a source can be used herein. The legumes include, but are not limited to, chickpeas, garbanzo beans, lentils, peas, green peas, kidney beans, black beans, soy beans, pinto beans, navy beans, faba beans, peanuts, and the like.

In some embodiments, starting materials for plant protein extraction may be faba bean pulse flour and the like. In order to prepare the starting materials, faba bean pulses undergo pre-treatment steps such as cleaning, drying, sorting, dehulling, and splitting that allow the detachment of the hulls and the cotyledons from whole pulses. This pre-treatment to legumes is well-known technique in the art.

### a) Protein Extraction

In some aspects, the PPI extraction process comprises a) extracting protein from a plant product in a basic aqueous solution. In some embodiments, the protein extracting step a) is performed salt. In other embodiments, the protein extracting step a) is performed comprising SHMP in the aqueous solution. When the a) protein extracting step comprises using SHMP it is called salt-induced protein extraction herein. When the a) protein extracting step does not include SHMP, it is called isoelectric protein extraction herein. In some cases, the isoelectric extraction step uses pH adjustment to extract protein.

In some embodiments the protein extracting step uses a basic aqueous solution comprising 0.2% to 5% SHMP. In certain embodiments the basic aqueous solution comprises 0.5% SHMP. In some embodiments the basic aqueous solution comprises 01.0% SHMP. In certain embodiments the basic aqueous solution comprises 0.1%, 0.2%, 0.3%, 0.4%, 0.6%, 0.7%, 0.8% or 0.9% SHMP. In certain embodiments the basic aqueous solution comprises 1.1%, 1.2%, 1.3%, 1.4%, 1.5%, 1.6%, 1.7%, 1.8%, 1.9% or 2.0% SHMP. In some embodiments the basic aqueous solution comprises 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%. 4.9% or 5.0% SHMP.

In some embodiments, the basic aqueous solution is a mixture of water and plant product which was prepared by one or more pretreatment steps, adjusted to a desired pH. In some embodiments the plant product is Faba bean flour. In some cases, the ratio of plant product to water is 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:25, 1:30. In certain cases the concentration of plant product in the basic aqueous solution is 2% to 40% solids. In some embodiments the concentration of plant product in the basic aqueous solution is 10%. In certain embodiments the concentration of plant product in the basic aqueous solution is 1%, 2%, 3% 4%, 5%, 6%, 7%, 8%, or 9%. In some embodiments the concentration of plant product in the basic aqueous solution is 11%, 12%, 13%, 14%, 15%, 16%, 17%, 18%, 19%, 20%, 20%, 22%, 23%, 24%, 25%, 26%, 27%, 28%, 29%, 30%, 31%, 32%, 33%, 34%, 35%, 36%, 37%, 38%, 239% or 40%.

In some embodiments, the protein extracting step a) is performed by regulating pH. In some embodiments, the protein extraction of step a) is performed at a pH of about 7 to 10. In certain embodiments, pH of the salt extracting of step a) is in the range of pH 7 to 9.5, pH 7 to 9, pH 7 to 8.5, pH 7 to 8, pH 7 to 7.5, pH 7.5 to 10, pH 7.5 to 9.5, pH 7.5 to 9, pH 8 to pH 9.5, or pH 8.5 to 9.5. In certain cases, pH of the salt extracting of step a) is in the range of pH 7 to 9.5.

In some embodiments, the protein extracting step a) is performed at a temperature of about 15° C. to about 85° C. In certain embodiments, the temperature for the salt extraction of step a) is in the range of about 20 to about 80° C., about 25 to about 80° C., about 30 to about 80° C., about 35 to about 80° C., about 40 to about 80° C., about 45 to about 80° C., about 50 to about 80° C., about 55 to about 80° C., about 60 to about 80° C., about 65 to about 95° C., about 70 to about 95° C., about 50 to about 90° C., about 50 to about 85° C., about 50 to about 80° C., about 50 to about 75° C., about 50 to about 70° C., about 60 to about 85° C., about 60 to about 80° C., about 60 to about 75° C., about 60 to about 80° C., about 60 to about 75° C., about 18 to about 60° C., about 18 to about 50° C., about 18 to about 40° C., or about 18 to about 35° C. In certain cases, the temperature for the salt extracting of step a) is in the range of about 55 to about 60° C. In some embodiments the protein extracting step is performed at 55° C.

In some embodiments, the time for the protein extracting of step a) is in the range of about 10 to 60 minutes for extraction. In certain embodiments, the time for the protein extracting step a) is about 15 minutes, about 20 minutes, about 25 minutes, about 30 minutes, about 35 minutes, about 40 minutes, about 45 minutes, about 50 minutes, about 55 minutes, or about 60 minutes.

b) Separating the Protein Solution from Unsolublized Plant Solids

In some aspects, the plant protein isolate (PPI) extraction process further comprises b) separating plant solids from the protein solution. In certain embodiments, the liquid fraction from step b) is supernatant. For example, the liquid fraction includes protein, mineral, vitamin, and the like.

In some embodiments, the separating of step b) is performed by centrifugation. The solids are then separated from the aqueous component, producing a protein solution for further processing. Means for separating solids from aqueous component are well known in the art. A common method of separation is centrifugation. In certain embodiments, the centrifugation is performed at 3000 g to 7000 g. In some embodiments the centrifugation is performed at 5000 g.

In some embodiments the centrifugation is performed for about 5 to about 30 minutes. In certain embodiments the centrifugation is performed for about 6, about 7, about 8, or about 9 minutes. In some embodiments thew centrifugation is performed for about 10 minutes. In certain embodiments the centrifugation is performed for about 11, about 12, about 13, about 14, about 15, about 16, about 17, about 18, about 19, about 20, about 21, about 22, about 23, about 24, about 25, about 26, about 27, about 28, about 29 or about 30 minutes.

In some embodiments the centrifugation is performed at room temperature. In certain embodiments the centrifugation is performed at a temperature of about 15° C. to about 60° C. In some embodiments the centrifugation is performed at about 25° C. In certain embodiments the centrifugation is performed at a temperature of about 16° C., about 17° C., about 18° C., about 19° C., about 20° C., about 21° C., about 22° C., about 23° C., about 24° C., about 26° C., about 27° C., about 28° C., about 29° C., about 30° C., about 31° C., about 32° C., about 33° C., about 34° C., about 35° C., about 36° C., about 37° C., about 39° C. or about 40° C. In some embodiments the centrifugation is performed at about 41° C., about 42° C., about 43° C., about 44° C., about 45, about 46° C., about 47° C., about 48° C., about 49° C., about 50° C., about 51° C., about 52° C., about 53° C., about 54° C., about 55° C., about 56° C., about 57° C., about 58° C., about 59° C. to about 60° C.

In other embodiments, the separation of step b) is performed by decanting. Any method or combination of methods of separation may be employed, and the determination of a proper method of separating a liquid fraction from the solids is well within the skill of the ordinary artisan.

### c) Precipitation of Protein

In some aspects, the PPI extraction process comprises steps of a) extracting protein from plant product in a basic aqueous solution comprising SHMP to produce a protein solution, b) separating solids from the protein solution and c) precipitating protein by lowering pH, thereby producing a PPI.

In certain embodiments the PPI extraction process comprises steps of a) extracting protein from plant product in a basic aqueous solution produce a protein solution, b) separating solids from the protein solution and c) precipitating protein by adding SHMP and lowering pH, thereby producing a PPI. In such embodiments, the protein precipitating step uses 0.2% to 5% SHMP. In certain embodiments the protein precipitating step uses 0.5% SHMP. In some embodiments the protein precipitating step uses 01.0% SHMP. In certain embodiments the protein precipitating step uses 0.1%, 0.2%, 0.3%, 0.4%, 0.6%, 0.7%, 0.8% or 0.9% SHMP. In certain embodiments the protein precipitating step uses 1.1%, 1.2%, 1.3%, 1.4%, 1.5%, 1.6%, 1.7%, 1.8%, 1.9% or 2.0% SHMP. In some embodiments the protein precipitating step uses 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%. 4.9% or 5.0% SHMP.

In some embodiments, the precipitating protein of step is performed by reducing the pH to about 3 to about 7. In certain embodiments, pH of the precipitating protein of step d) is reduced to the range of pH 4 to 7.5, pH 4 to 7, pH 5 to 8, pH 5 to 7.5, or pH 5 to 7.

In some embodiments, the precipitation of protein of step is performed for 30 minutes to 60 minutes. In certain embodiments, precipitating protein is performed for about 30 minutes, about 35 minutes, about 40 minutes, about 45 minutes, about 50 minutes, about 55 minutes, or about 60 minutes. in the range of about 10 to 60 minutes for extraction. In certain embodiments, the time for the precipitating protein e step c) is about 15 minutes, about 20 minutes, about 25 minutes, about 30 minutes, about 35 minutes, about 40 minutes, about 45 minutes, about 50 minutes, about 55 minutes, or about 60 minutes.

In some embodiments, the precipitating of protein step c) is performed at a temperature in the range of about 15 to about 80° C., about 18 to about 70° C., about 20 to about 60° C., about 22 to about 55° C., about 20 to about 60° C., about 20 to about 65° C., about 25 to about 60° C., about 30 to about 60° C., about 30 to about 55° C., about 25 to about 60° C., about 25 to about 50° C., about 30 to about 60° C., about 30 to about 55° C. In certain embodiments the precipitating step is performed at about 55° C.

d) Washing PPI from Step c)

In some aspects, the albumin enriched PPI extraction process further comprises a step of d) washing the PPI from step c). In some embodiments, the washing of step d) is performed with water at the precipitation pH, such as pH about 3.5 to about 7. In other embodiments, the protein isolate from step d) is washed two times at precipitation pH such as pH in the range of about 3.7 to about 7, pH 4 to 7, pH 4 to 6.5, pH 3.5 to 6, pH 4 to 5.5, or pH 4.5 to 5. The washing step is a well-known technique in the art. In certain embodiments the washing is performed at about pH 4.5.

### e) Diluting the PPI to Make Slurry

In other aspects, the PPI extraction process further comprises a step of e) diluting the PPI to make a protein slurry. Creating a protein slurry is well-known technique in the art.

In some aspects, the PPI extraction process comprises a step of diluting the protein slurry with water and adjusting its pH to neutral. In certain embodiments the pH is adjusted to about pH 7. The dilution process is a well-known technique in the art.

### f) Homogenizing and Sterilizing the Protein Slurry

In some aspects, the plant protein isolate (PPI) extraction process further comprises f) homogenizing the PPI. In certain embodiments the process includes sterilizing PPI. In some embodiments, the protein slurry is homogenized by means known in the art. For example, a high-pressure homogenizer may be used. The homogenized protein slurry may then be sterilized and/or pasteurized.

Means for sterilizing protein for use in foods are known in the art. In some embodiments, the sterilization temperature is in the range of 75 to 140° C., 80 to 140° C., 90 to 140° C., 100 to 140° C., 75 to 130° C., 75 to 120° C., 75 to 110° C., or 75 to 100° C. An example of such a means is direct steam injection. The protein slurry may be subjected to direct steam injection for between 2 seconds and 10 minutes, such as 2 seconds, 5 seconds, 10 seconds, 15 seconds, 20 seconds, 25 seconds, 30 seconds, 35 seconds, 40 seconds, 45 seconds, 50 seconds, 55 seconds, 1 minutes, 2 minutes, 3 minutes, 4 minutes, 5 minutes, 6 minutes, 7 minutes, 8 minutes, 9 minutes, 10 minutes.

### h) Drying the PPI

In some aspects, the Plant protein isolate (PPI) extraction process further comprises drying the PPI. The PPI is dried using a drying apparatus known in the art, provided that heat above about 60° C. is not used. Several such apparatuses, such as spray dryers, freeze dryer, ring dryers, dispersion dryers, drum dryers and fluid bed dryers, are commercially available. The resulting plant protein isolate may then be used as further described below.

In certain aspects, the PPI extraction process has improved protein yield by about 8 to about 10% as compared with the conventional PPI extraction process.

### Plant Protein Isolates (PPI)

In addition to the PPI extraction process, the present disclosure provides PPIs produced by such processes of the present disclosure and PPIs having particular useful characteristics. The PPI extraction process is described in the section “Plant Protein Isolate Extraction Process” above. The plant protein isolates are referred to herein as PPI.

In some embodiments, the plant protein isolate produced by the above process is faba bean protein isolate FPI. In some embodiments, the PPI produced by the above process has a protein content in the range of about 80 to about 93 wt % (N×6.25) on a dry basis. In other embodiments, the PPI has protein content in the range of about 80% to about 95% on a dry basis. In yet other embodiments, the PPI has protein content in the range of about 80% to about 98% on a dry basis. The process allows for the protein of the albumin enriched PPIs to substantially maintain its native form.

In some embodiments, the PPI produced by the above process has a unique composition.

In some embodiments, the PPI produced by the above process has unique functionality as compared with conventional PPI. Such improved functionality may be, but is not limited to, improved foaming ability, improved emulsify ability, improved gelling ability, and improved solubility.

In some embodiments, the PPI has protein solubility improved by at least 30% as compared with conventional PPI. In certain embodiments, the PPI has protein solubility improved by at least 35%, 40%, 45%, 50%, 55%, 60%, 65%, 70%, 75%, 80%, 85%, 90% as compared with conventional PPI.

In certain embodiments the PPI is FPI. In some embodiments the FPI has 80-90% protein solubility at room temperature and pH 7. In some embodiments the FPI has >80% protein solubility at room temperature and pH 7. In some embodiments the FPI has 82-90% protein solubility at room temperature and pH 7. In certain embodiments the PPI is CPI. In some embodiments the CPI has >70% protein solubility at room temperature and pH 7. In some embodiments the CPI has 73-80% protein solubility at room temperature and pH 7. In certain embodiments the PPI is MPI. In some embodiments the MPI has 80-85% protein solubility at room temperature and pH 7. In some embodiments the MPI has >80% protein solubility at room temperature and pH 7. In certain embodiments the PPI is LPI. In some embodiments the LPI has 80-85% protein solubility at room temperature and pH 7. In some embodiments the LPI has >80% protein solubility at room temperature and pH 7. In certain embodiments the PPI is UPI. In some embodiments the UPI has 82-87% protein solubility at room temperature and pH 7. In some embodiments the UPI has >80% protein solubility at room temperature and pH 7. In certain embodiments the PPI is PePI. In some embodiments the PePI has 90-92% protein solubility at room temperature and pH 7. In some embodiments the PePI has >90% protein solubility at room temperature and pH 7.

In some embodiments protein in the PPI is substantially in native form. In certain embodiments the PPI has improved thermodynamic characteristics compared with conventional PPI.

In some embodiments, the FPI has thermostability to about 97° C. In certain embodiments the FPI has an improved color profile compared with conventional FPI.

In some embodiments of the invention, the FPI has improved foaming expansion capacity compared with conventional FPI. In certain embodiments, the FPI of the present invention has >200% foaming expansion capacity (% FE). In some embodiments the FPI % FE of about 400% when mixed with water (5% w/w) and mixed with a homogenizer at 10000 rpm for 1 minute. In certain embodiments the FPI has improved foaming stability compared with conventional FPI. In some embodiments the FPI has 70% foaming stability (% FS) after 1 hour when mixed with a homogenizer at 10000 rpm for 1 minute.

### Use of the Plant Protein Isolates (PPI)

In some aspects, the present disclosure provides use of the PPI of the present disclosure in the production of a foam. In certain embodiments, the present disclosure provides use of the FPI of the present disclosure in the production of a foam.

In other aspects, the present invention provides use of the PPI of the present disclosure in the production of a gel. In some embodiments, the present invention provides use of the FPI of the present disclosure in the production of a gel.

In still other aspects, the present invention provides use of the PPI of the present disclosure in the production of an emulsion. In certain embodiments, the present invention provides use of the FPI of the present disclosure in the production of an emulsion. In some embodiments, the emulsion is made from a mixture of equal parts oil and water.

In some aspects, the present disclosure provides a foam comprising the PPI of the present disclosure. In certain embodiments, the present disclosure provides a foam comprising the FPI of the present disclosure

In other aspects, the present disclosure provides a gel comprising the PPI. In certain embodiments, the present disclosure provides a gel comprising the FPI.

In still other aspects, the present disclosure provides an emulsion comprising the PPI of the present disclosure. In certain embodiments, the present disclosure provides an emulsion comprising the FPI of the present disclosure.

In some aspects, the present disclosure provides use of the PPI of the present disclosure in a food or beverage application. In certain embodiments, the present disclosure provides use of the FPI of the present disclosure in a food or beverage application. In some embodiments, the food or beverage application is selected from milk shake, protein bars, meat analogues, confectionary, condiments, mayonnaise, salad dressing, nutritional supplements and diary alternatives. In certain embodiments, the dairy alternative is selected from creamers, ice cream, yogurt, buttermilk and cheese.

In yet other aspects, the present disclosure provides a food or beverage comprising the PPI of the present disclosure. In certain embodiments, the present disclosure provides a food or beverage comprising the FPI of the present disclosure.

### Exemplary Non-limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-88 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A process for producing a plant protein isolate comprising:
   a. extracting protein from a plant product in a basic aqueous solution comprising 0.2-1.5% sodium hexametaphosphate (SHMP) to create a protein solution;
   b. separating the protein solution from unsolublized plant solids;
   c. precipitating protein from the protein solution by lowering pH;
to produce a plant protein isolate.
2. A process for producing a plant (faba been) protein isolate comprising:
   a. extracting protein from a plant product in a basic aqueous solution to create a protein solution;
   b. separating the protein solution from unsolublized plant solids;
   c. precipitating protein from the protein solution of by:
      i. reducing the pH of the protein solution, and
      ii. adding 0.2% to 1.5% SHMP,
to produce a plant protein isolate.
3. The process of aspect 1 or aspect 2, wherein said extracting is performed at a pH of 7.0 to 9.5.
4. The process of aspect 1, wherein said extracting is performed at pH 7.5.
5. The process of aspect 2, wherein said extracting is performed at pH 8.0.
6. The process of aspect 1 or aspect 2 wherein said extracting is performed for 10-60 minutes.
7. The process of aspect 1 or aspect 2 wherein said extracting is performed for 30 minutes.
8. The process of aspect 1 or aspect 2 wherein said extracting is performed at a temperature of 30-80° C.
9. The process of aspect 1 or aspect 2 wherein said extracting is performed at a temperature of 55° C.
10. The process of aspect 1, wherein said extracting is performed in a solution comprising 0.5% SHMP.
11. The process of aspect 1 or aspect 2 wherein said extract is performed in a solution at an initial concentration of 2%-40% solids.
12. The process of aspect 1 or aspect 2 wherein said extract is performed in a solution at an initial concentration of 10% solids.
13. The process of aspect 1 or aspect 2 wherein said separating is performed by centrifugation.
14. The process of aspect 13, wherein said centrifugation is performed at 3000 g to 7000 g.
15. The process of aspect 13, wherein said centrifugation is performed at 5000 g.
16. The process of aspect 13, wherein said centrifugation is performed for 5-30 minutes.
17. The process of aspect 13, wherein said centrifugation is performed for 10 minutes.
18. The process of aspect 13, wherein said centrifugation is performed at a temperature of 15-60° C.
19. The process of aspect 13, wherein said centrifugation is performed at a temperature of 25° C.
20. The process of aspect 13, wherein said centrifugation is performed at room temperature.
21. The process of aspect 1 or aspect 2 wherein said separating is by decanting.
22. The process of aspect 1 or aspect 2, wherein said precipitating is performed at pH 3 to pH 7.
23. The process of aspect 1 or aspect 2, wherein said precipitating is performed at pH 4.5.
24. The process of aspect 2 wherein said precipitating is performed in the presence of 0.2% to 1.5% SHMP.
25. The process of aspect 2 wherein said precipitating is performed in the presence of 0.5% SHMP.
26. The process of aspect 2 wherein said precipitating is performed in the presence of 1% SHMP.
27. The process of aspect 2 wherein said precipitating is performed in the presence of 0.5% SHMP, increased to 1% SHMP during precipitation.
28. The process of aspect 1 or aspect 2, wherein said precipitating is performed at a temperature of 30-80° C.
29. The process of aspect 1 or aspect 2, wherein said precipitating is performed at a temperature of 55° C.
30. The process of aspect 1 or aspect 2 wherein said precipitating is performed for 10-60 minutes.
31. The process of aspect 1 or aspect 2 wherein said precipitating is performed for 30 minutes.
32. The process of aspect 1 or aspect 2, further comprising: d. washing said plant protein isolate.
33. The process of aspect 32, wherein said washing is with water.
34. The process of aspect 32, wherein said washing is at precipitating pH.
35. The process of any of aspects 32-34, wherein said washing is performed 2 times.
36. The process of any one of aspects 1-35, further comprising: e. diluting the protein isolate to make a slurry.
37. The process of aspect 35, wherein said diluting is with water.
38. The process of any one of aspects 36-37, wherein said diluting further comprises adjusting the pH of said slurry to neutral.
39. The process of aspect 38 wherein said adjusting the pH of the slurry is to pH 7.
40. The process of any one of aspects 1-39, further comprising: f. homogenizing the slurry.
41. The process of aspect 40 wherein said homogenization is performed using a high pressure homogenizer.
42. The process of any one of aspects 1-41, further comprising: g sterilizing and/or pasteurizing the protein isolate.
43. The process of aspect 42, wherein said sterilizing is performed by direct stream injection.
44. The process of aspect 42 or 43 wherein said sterilizing is performed at 75-140° C.
45. The process of any one of aspects 42-44, wherein said sterilizing is performed for 2 seconds to 10 minutes.
46. the process of any one of aspects 1-45, further comprising: h. drying the protein isolate.
47. The process of aspect 46 wherein said drying is performed using a freeze/spray drier.
48. A plant protein isolate produced by the process of aspect 1 or aspect 2.
49. A plant protein isolate produced by the process of any one of aspects 1-47.
50. The plant protein isolate of aspect 48 which is a faba bean protein isolate (FPI).
51. The plant protein isolate of aspect 49 which is a faba bean protein isolate (FPI).
52. The FPI of aspect 50 or 51 comprising native protein structure.
53. FPI having thermal stability to about 97° C.
54. FPI having a thermal transition with a peak at about 96.6° C., as measured by differential scanning calorimetry (DSC).
55. FPI having a thermal transition with an onset of about 83.8° C., a peak at about 96.6° C. and an offset of about 107.3° C., as measured by DSC.
56. The FPI of aspect 50 or 51 having an improved color profile as compared with PPI produced using conventional processes.
57. An emulsion comprising the FPI of aspect 50 or 51.
58. Use of the FPI of aspect 50 or 51 in the production of an emulsion.
59. FPI having 100% emulsion stability (ES) in cold storage after 5 days at a concentration of 2-10% (w/w) in a 50:50 oil in water emulsion.
60. FPI having 100% ES in cold storage after 5 days at a concentration of 5% (w/w) in a 50:50 oil in water emulsion.
61. A foam comprising the FPI of aspect 50 or 51.
62. Use of the FPI of aspect 50 or 51 in the production of a foam.
63. FPI having about 400% foam expansion capacity (FE) at a concentration of 2-10% (w/w) in water when homogenized at 1000 rpm for 1 minute.
64. FPI having about 400% FE at a concentration of 5% (w/w) in water when homogenized at 1000 rpm for 1 minute.
65. FPI having about 70% foaming stability (FS) after 1 hour at a concentration of 2-10% (w/w) in water when homogenized at 1000 rpm for 1 minute.
66. FPI having about 70% Fs after 1 hour at a concentration of 5% (w/w) in water when homogenized at 1000 rpm for 1 minute.
67. FPI having protein solubility of 80-92%.
68. FPI having protein solubility of 82-90%.
69. FPI having percent solubility in water at room temperature and pH 7 of >80%.
70. The plant protein isolate of aspect 48 which is a mung bean protein isolate (MPI).
71. The plant protein isolate of aspect 49 which is a mung bean protein isolate (MPI).
72. MPI having percent solubility in water at room temperature and pH 7 of >80%.
73. The plant protein isolate of aspect 48 which is a chickpea protein isolate (CPI).
74. The plant protein isolate of aspect 49 which is a chickpea protein isolate (CPI).
75. CPI having percent solubility in water at room temperature and pH 7 of >70%.
76. The plant protein isolate of aspect 48 which is a lentil protein isolate (LPI).
77. The plant protein isolate of aspect 49 which is a lentil protein isolate (LPI).
78. LPI having percent solubility in water at room temperature and pH 7 of >80%.
79. The plant protein isolate of aspect 48 which is a black gram protein isolate (UPI).
80. The plant protein isolate of aspect 49 which is a black gram protein isolate (UPI).
81. UPI having percent solubility in water at room temperature and pH 7 of >80%.
82. The plant protein isolate of aspect 48 which is a pea protein isolate (PePI).
83. The plant protein isolate of aspect 49 which is a pea protein isolate (PePI).
84. PePI having percent solubility in water at room temperature and pH 7 of >90%.
85. The process of aspect 1 or 2, wherein the plant product is a pulse.
86. The process of any of aspects 1-47, wherein the plant product is a pulse.
87. The process of aspect 85, wherein the pulse is derived from a plant selected from a faba bean, a mung bean, a chickpea, a lentil, a black gram and a pea.
88. The process of any of aspects 1-47, wherein the plant product is derived from a plant selected from a faba bean, a mung bean, a chickpea, a lentil, a black gram and a pea.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, temperature is in degrees Centigrade, and times are in minutes.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

### Process

In this Example, FPI was prepared from faba bean flour utilizing SHMP in the protein extraction step (step 1).

TABLE 1

| No. | Extraction | Process details |
|---|---|---|
| 1 | Aqueous/Salt | pH of extraction: 7.5 |
| | Extraction | Time of extraction: 30 min |
| | | Temperature of extraction: 55° C. |
| | | SHMP: 0.5% |
| | | Concentration: 10% plant solids |
| 2 | Centrifugation | 5000 g for 10 minutes at room temperature |
| 3 | Precipitation of | Precipitation of protein at pH 4.5 for 30 min |
| | protein | Precipitation temperature: 55° C. |
| 4 | Washing of | Washing the protein with 2x water at |
| | protein isolate | precipitation pH |
| | from step 3 | |
| 5 | Dilution | Diluting the protein slurry with 2x water and |
| | | adjust pH to pH 7 |
| 6 | Homogenization | Homogenizing the protein slurry using a high- |
| | and Sterilization | pressure homogenizer |
| | | Sterilizing the protein slurry in a direct steam |
| | | injection |
| 7 | Drying | Drying the protein slurry by a freeze/spray drier |

### Results

The FPI has a protein content in the range of 90-93% on a dry basis.

The above extraction process enhanced the protein yield 8-10% and enhanced the solubility in water at room temperature an pH 7 of FPI by 40% compared to of conventional FPI, as shown in Table 2.

TABLE 2

| Sample | Protein solubility (%) |
|---|---|
| FPI | 82-90 |
| CONV FPI | 45-50 |

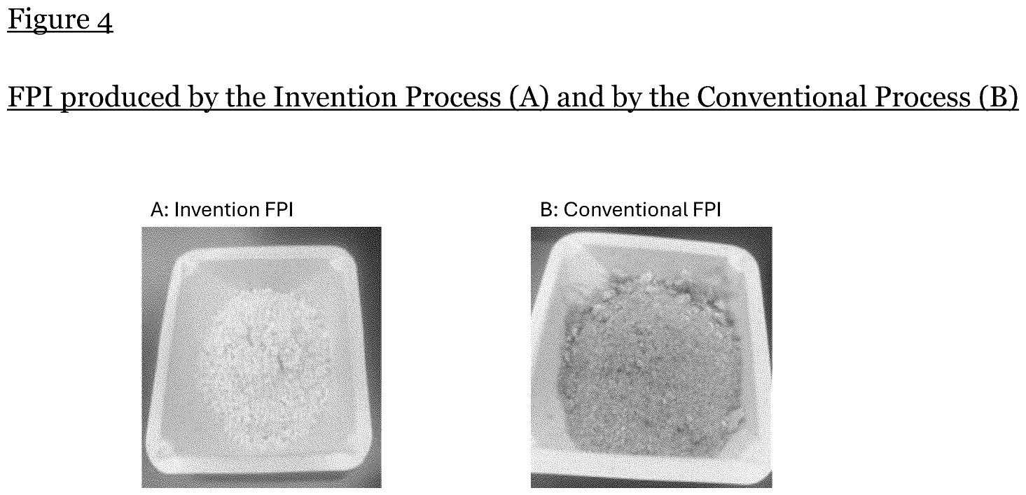

The difference in color profile of the FPI and conventional FPI are shown in FIGS. 4A and 4B, respectively. The FPI of the invention presents a significantly more appealing color.

### Example 2

### DSC Analysis

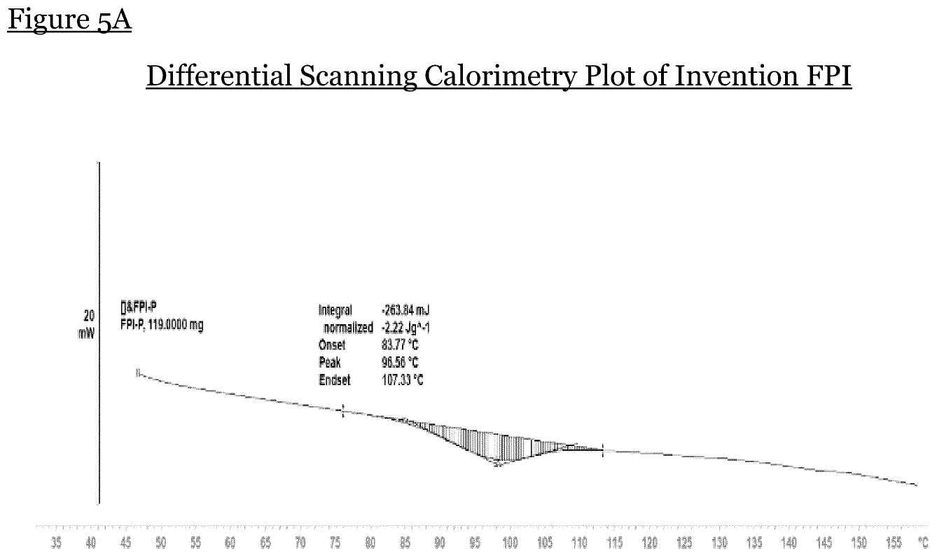

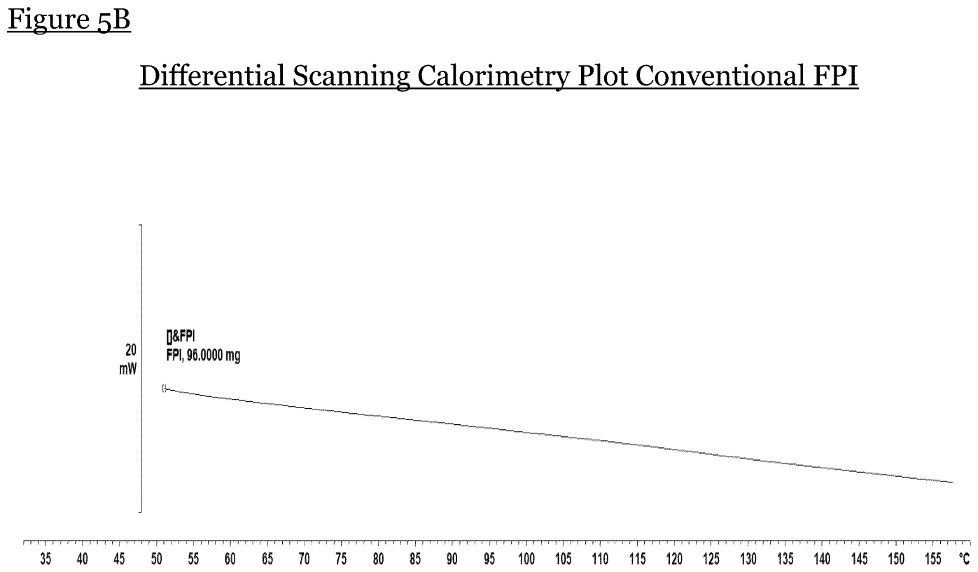

The thermal properties of FPI of the invention compared to conventional FPI were analyzed differential scanning calorimetry (DSC). FIGS. 5A and 5B show the thermal profile of FPI of the invention and conventional FPI, respectively. The FPI of the invention shows stability to ˜97° C. The thermal transition data indicates that in FPI protein structure is in substantially native form and function is preserved compared to the conventional FPI. The thermal profile of the FPI and conventional FPI are summarized in Table 3.

TABLE 3

| | Transition | Transition | Transition | |
|---|---|---|---|---|
| | Onset | Peak | Offset | Enthalpy |
| Sample | (° C.) | (° C.) | (° C.) | (J/g) |
| FPI | 83.8 | 96.6 | 107.3 | 2.22 |
| Conv. FPI | — | — | — | — |

### Example 3

### Emulsions

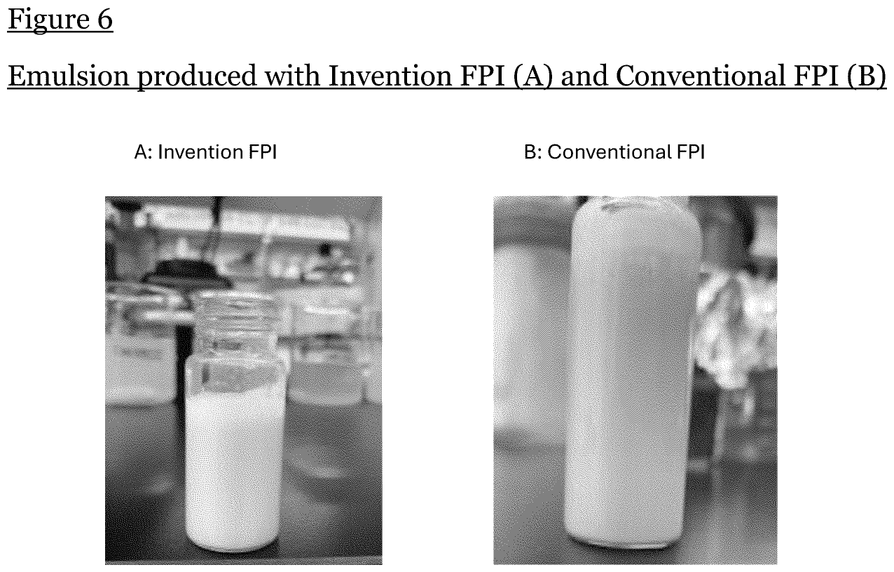

Formation of an emulsion and emulsion stability were tested for FPI of the invention and conventional FPI. Conventional FPI and FPI of the invention were used to make emulsions in 50/50 oil and water at pH 7. FPI (5% w/w) was mixed in the oil and water using an Ultra-Turrax homogenizer run at 8000 rpm for 2 minutes. FIGS. 6A and 6B show emulsions made using the FPI and conventional FPI, respectively. The FPI of the invention showed 100% emulsion stability after 5 days in cold storage. In contrast, the conventional FPI showed 100% emulsion stability in cold storage for only 24 hours.

### Example 4

### Foams

Foams were made with FPI of the invention and conventional FPI in water (5% w/w) using an Ultra-Turrax homogenizer run at 10000 rpm for 1 minute. Percent foaming expansion capacity (% FE) and percent foaming stability (% FS) were determined. Percent foaming expansion (% FE) is determined using the equation % FE=(Vfo/Vli)×100, where Vfo is the measured volume of agitated solution and Vli is the measured initial volume of liquid before agitation. Percent foaming stability (% FS) is measured using the equation % FS=[(Vli−Vlt)/(Vli−Vlo)], where Vli is the measured initial volume of protein solution before agitation, Vlt is the volume of the protein solution one hour after agitation and Vlo is the volume of the protein solution immediately after agitation.

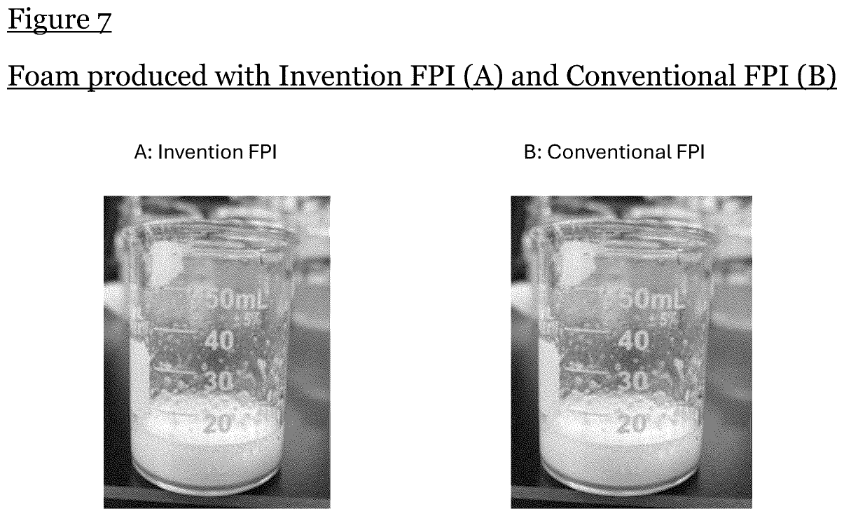

Foams made with FPI of the invention and conventional FPI are shown in FIGS. 7A and 7B, respectively. The FPI of the invention showed % FE of 400% while conventional FPI Showed % FE of 200%. FPI also showed % FS of 70% while conventional FPI showed % FS of 50%.

### Example 5

### Process

In this Example, detailed in Table 4, FPI was prepared from faba bean pulse flour utilizing SHMP in the protein precipitation step (step 3).

TABLE 4

| No. | Extraction | Process details |
|---|---|---|
| 1 | Aqueous/Salt | pH of extraction: 8.0 |
| | Extraction | Time of extraction: 30 min |
| | | Temperature of extraction: 55° C. |
| | | Concentration: 10% plant solids |
| 2 | Centrifugation | 5000 g for 10 minutes at room temperature |
| 3 | Precipitation of | Precipitation of protein at pHs 4.5 for 30 min |
| | protein | SHMP: 0.5% and 1% |
| | | Precipitation temperature: 55° C. |
| 4 | Washing of | Washing the protein with 2x water at |
| | protein isolate | precipitation pHs |
| | from step 3 | |
| 5 | Dilution | Diluting the protein slurry with 2x water and |
| | | adjust its pH to pH 7 |
| 6 | Homogenization | Homogenizing the protein slurry using a high- |
| | and Sterilization | pressure homogenizer |
| | | Sterilizing the protein slurry in a direct steam |
| | | injection |
| 7 | Drying | Drying the protein slurry by a freeze/spray drier |

The resulting products had similar solubility attribute to the FPI produced using the process described in Example 1. In addition, no detectable difference in solubility was found between FPI produced using 0/5% SHMP and 1% SHMP.

### Example 6

In this Example, mung bean protein isolate (MPI) was prepared from mung bean (Vigna radiata) flour utilizing SHMP in the protein extraction step (step 1).

TABLE 1

| No. | Extraction | Process details |
|---|---|---|
| 1 | Aqueous/Salt | pH of extraction: 7.5 |
| | Extraction | Time of extraction: 30 min |
| | | Temperature of extraction: 55° C. |
| | | SHMP: 0.5% |
| | | Concentration: 10% plant solids |
| 2 | Centrifugation | 5000 g for 10 minutes at room temperature |
| 3 | Precipitation of | Precipitation of protein at pH 4.5 for 30 min |
| | protein | Precipitation temperature: 55° C. |
| 4 | Washing of | Washing the protein with 2x water at |
| | protein isolate | precipitation pH |
| | from step 3 | |
| 5 | Dilution | Diluting the protein slurry with 2x water and |
| | | adjust pH to pH 7 |
| 6 | Homogenization | Homogenizing the protein slurry using a high- |
| | and Sterilization | pressure homogenizer |
| | | Sterilizing the protein slurry in a direct steam |
| | | injection |
| 7 | Drying | Drying the protein slurry by a freeze/spray drier |

### Results

The MPI had protein content of 86% on a dry basis. The solubility of the MPI in water at pH 7 was 83%.

### Example 7

In this Example, chickpea protein isolate (CPI) was prepared from chickpea (Cicer arietum) flour utilizing SHMP in the protein extraction step (step 1).

TABLE 1

| No. | Extraction | Process details |
|---|---|---|
| 1 | Aqueous/Salt | pH of extraction: 7.5 |
| | Extraction | Time of extraction: 30 min |
| | | Temperature of extraction: 55° C. |
| | | SHMP: 0.5% |
| | | Concentration: 10% plant solids |
| 2 | Centrifugation | 5000 g for 10 minutes at room temperature |
| 3 | Precipitation of | Precipitation of protein at pH 4.5 for 30 min |
| | protein | Precipitation temperature: 55° C. |
| 4 | Washing of | Washing the protein with 2x water at |
| | protein isolate | precipitation pH |
| | from step 3 | |
| 5 | Dilution | Diluting the protein slurry with 2x water and |
| | | adjust pH to pH 7 |
| 6 | Homogenization | Homogenizing the protein slurry using a high- |
| | and Sterilization | pressure homogenizer |
| | | Sterilizing the protein slurry in a direct steam |
| | | injection |
| 7 | Drying | Drying the protein slurry by a freeze/spray drier |

### Results

The CPI had protein content of 86% on a dry basis. The solubility of the CPI in water at pH 7 was 74%.

### Example 8

In this Example, lentil protein isolate (LPI) was prepared from lentil (Lens culinaris) flour utilizing SHMP in the protein extraction step (step 1).

TABLE 1

| No. | Extraction | Process details |
|---|---|---|
| 1 | Aqueous/Salt | pH of extraction: 7.5 |
| | Extraction | Time of extraction: 30 min |
| | | Temperature of extraction: 55° C. |
| | | SHMP: 0.5% |
| | | Concentration: 10% plant solids |
| 2 | Centrifugation | 5000 g for 10 minutes at room temperature |
| 3 | Precipitation of | Precipitation of protein at pH 4.5 for 30 min |
| | protein | Precipitation temperature: 55° C. |
| 4 | Washing of | Washing the protein with 2x water at |
| | protein isolate | precipitation pH |
| | from step 3 | |
| 5 | Dilution | Diluting the protein slurry with 2x water and |
| | | adjust pH to pH 7 |
| 6 | Homogenization | Homogenizing the protein slurry using a high- |
| | and Sterilization | pressure homogenizer |
| | | Sterilizing the protein slurry in a direct steam |
| | | injection |
| 7 | Drying | Drying the protein slurry by a freeze/spray drier |

### Results

The LPI had protein content of 88% on a dry basis. The solubility of the LPI in water at pH 7 was 81%.

### Example 9

In this Example, black gram protein isolate (UPI) was prepared from black gram (Vigna mungo) flour utilizing SHMP in the protein extraction step (step 1).

TABLE 1

| No. | Extraction | Process details |
|---|---|---|
| 1 | Aqueous/Salt | pH of extraction: 7.5 |
| | Extraction | Time of extraction: 30 min |
| | | Temperature of extraction: 55° C. |
| | | SHMP: 0.5% |
| | | Concentration: 10% plant solids |
| 2 | Centrifugation | 5000 g for 10 minutes at room temperature |
| 3 | Precipitation of | Precipitation of protein at pH 4.5 for 30 min |
| | protein | Precipitation temperature: 55° C. |
| 4 | Washing of | Washing the protein with 2x water at |
| | protein isolate | precipitation pH |
| | from step 3 | |
| 5 | Dilution | Diluting the protein slurry with 2x water and |
| | | adjust pH to pH 7 |
| 6 | Homogenization | Homogenizing the protein slurry using a high- |
| | and Sterilization | pressure homogenizer |
| | | Sterilizing the protein slurry in a direct steam |
| | | injection |
| 7 | Drying | Drying the protein slurry by a freeze/spray drier |

### Results

The UPI had protein content of 89% on a dry basis. The solubility of the UPI in water at pH 7 was 84%.

### Example 10

In this Example, pea protein isolate (PePI) was prepared from pea (Pisum sativum) flour utilizing SHMP in the protein extraction step (step 1).

TABLE 1

| No. | Extraction | Process details |
|---|---|---|
| 1 | Aqueous/Salt | pH of extraction: 7.5 |
| | Extraction | Time of extraction: 30 min |
| | | Temperature of extraction: 55° C. |
| | | SHMP: 0.5% |
| | | Concentration: 10% plant solids |
| 2 | Centrifugation | 5000 g for 10 minutes at room temperature |
| 3 | Precipitation of | Precipitation of protein at pH 4.5 for 30 min |
| | protein | Precipitation temperature: 55° C. |
| 4 | Washing of | Washing the protein with 2x water at |
| | protein isolate | precipitation pH |
| | from step 3 | |
| 5 | Dilution | Diluting the protein slurry with 2x water and |
| | | adjust pH to pH 7 |
| 6 | Homogenization | Homogenizing the protein slurry using a high- |
| | and Sterilization | pressure homogenizer |
| | | Sterilizing the protein slurry in a direct steam |
| | | injection |
| 7 | Drying | Drying the protein slurry by a freeze/spray drier |

### Results

The PePI had protein content of 86% on a dry basis. The solubility of the PePI in water at pH 7 was 83%.

1. A process for producing a faba bean protein isolate comprising:
a. extracting protein from a faba bean product in a basic aqueous solution at pH 7.5 comprising 0.2-1.5% sodium hexametaphosphate (SHMP) to create a protein solution;
b. separating the protein solution from unsolubilized plant solids;
c. precipitating protein from the protein solution by lowering pH;
to produce a faba bean protein isolate having about 80 wt % to about 98 wt % protein content.

2. A process for producing a faba bean protein isolate comprising:
a. extracting protein from a faba bean product in a basic aqueous solution at pH 8 to create a protein solution;
b. separating the protein solution from unsolubilized faba bean solids;
c. precipitating protein from the protein solution of b. reducing the pH of the protein solution in the presence of 0.2% to 1.5% sodium hexametaphosphate (SHMP),
to produce a faba bean protein isolate having about 80 wt % to about 98 wt % protein content.

3-5. (canceled)

6. The process of claim 1 wherein said extracting is performed for 10-60 minutes.

7. (canceled)

8. The process of claim 1 wherein said extracting is performed at a temperature of 30-80° C.

9. (canceled)

10. The process of claim 1, wherein said extracting is performed in a solution comprising 0.5% SHMP.

11. The process of claim 1 wherein said extracting is performed in a solution at an initial concentration of 2%-40% solids.

12. (canceled)

13. The process of claim 1 wherein said separating is performed by centrifugation, at a temperature of 15-60° C.

14-21. (canceled)

22. The process of claim 1, wherein said precipitating is performed at pH 3 to pH 7 and at a temperature of 30-80° C. for 10-60 minutes.

23. (canceled)

24. (canceled)

25-31. (canceled)

32. The process of claim 1, further comprising:
d. washing said faba bean protein isolate, wherein said washing is at precipitating pH,
e. diluting the protein isolate to make a slurry, wherein said diluting is with water, and wherein said diluting further comprises adjusting the pH of said slurry to neutral,
f. homogenizing the slurry,
g. sterilizing or pasteurizing the protein isolate, and
h. drying the protein isolate.

33-47. (canceled)

48. A faba bean protein isolate produced by the process of claim 1.

49-88. (canceled)

89. (canceled)

90. The process of claim 2 wherein said extracting is performed for 10-60 minutes.

91. The process of claim 2 wherein said extracting is performed at a temperature of 30-80° C.

92. The process of claim 2 wherein said extracting is performed in a solution at an initial concentration of 2%-40% solids.

93. The process of claim 2 wherein said separating is performed by centrifugation at a temperature of 15-60° C.

94. The process of claim 2, wherein said precipitating is performed at pH 3 to pH 7 and at a temperature of 30-60° C. for 10-60 minutes.

95. The process of claim 2, further comprising:
d. washing said faba bean protein isolate, wherein said washing is at precipitating pH,
e. diluting the protein isolate to make a slurry, wherein said diluting is with water, and wherein said diluting further comprises adjusting the pH of said slurry to neutral,
f. homogenizing the slurry,
g. sterilizing or pasteurizing the protein isolate, and
h. drying the protein isolate.

96-97. (canceled)

98. A faba bean protein isolate produced by the process of claim 2.
